(19) United States
(12) Patent Application Publication
(10) Pub. No.: US 2025/0019606 A1
(43) Pub. Date: Jan. 16, 2025

(54) Process for the production of liquid fuels using biomass gasification integrated with an electrolyzer

(71) Applicant: Greyrock Technology, LLC, Sacramento, CA (US)

(72) Inventors: Glenn McGinnis, Sun Lakes, AZ (US); Robert Schuetzle, Sacramento, CA (US); Benjamin Wright, Brookfield, WI (US); Harold Wright, Menomonee Falls, WI (US)

(21) Appl. No.: 18/445,321

(22) Filed: Jul. 10, 2023

(51) Int. Cl. C10J 3/72; C07C 1/04; C07C 29/151; C10G 2/00; C10L 1/02; C10L 1/04

(52) U.S. Cl. CPC C10J 3/72; C10G 2/30; C10L 1/02; C10L 1/04; C10G 2300/1011; C10J 2300/0916; C10J 2300/1684; C10L 2200/0469

(57) ABSTRACT

An integrated process and system with processes configured to generate liquid fuels, electricity, and heat from carbonaceous fuel sources. The preferred system combines and maximizes the carbon and energy conversion potential and efficiency of the associated components or subsystems to produce a system for the co-production of fuels and electricity from syngas. The process can produce liquid fuels and electricity from carbonaceous feedstock at net thermal energy efficiencies of greater than 40-50%, which are significantly higher than fuel synthesis or electricity generation alone.

### FIELD OF THE INVENTION

The present invention is generally directed to the production of liquid hydrocarbon fuels from hydrogen (H₂) and carbon monoxide (CO) produced by the integration of biomass gasification with an electrolysis process and with a liquid fuel production process.

### BACKGROUND OF THE INVENTION

The worldwide demand for energy and transportation fuels is growing rapidly as fossil energy sources become more depleted, expensive, and environmentally problematic. A significant percentage of the production of electricity in the United States comes from conventional fossil-fuel-fired boilers linked to a conventional steam cycle. Such generation systems have modest efficiencies and contribute to the global emissions of nitrogen oxides, sulfur oxide, carbon dioxide, and particulate matter. Furthermore, in some fuel combustion plants, only a third of the energy value of the fuel is actually converted into electricity and the rest is lost as waste heat. Improved and innovative systems are needed that will provide alternative energy sources and options to meet future energy needs and address rising pollution issues.

### BRIEF SUMMARY OF THE INVENTION

In one aspect, an integrated system with processes configured to generate liquid fuels, electricity, and heat from carbonaceous fuel sources is provided. The preferred system combines and maximizes the carbon and energy conversion potential and efficiency of the associated components or subsystems to produce a system for the co-production of fuels and electricity from synthesis gas (“syngas” composed of primarily CO and H₂). The process can produce liquid fuels and electricity from carbonaceous feedstock at net thermal energy efficiencies of greater than 40-50%, which are significantly higher than fuel synthesis or electricity generation alone.

Some of the other advantages include: simplified process steps through the real-time monitoring of gas composition before and after the catalyst reactor; monitoring of process conditions (such as temperature, pressure and gasflow velocity); optimization of hydrogen/carbon monoxide gas composition using a hydrogen generator; the use of neural network algorithms and kinetic/thermodynamic models with feed-back control for process optimization; and the use of chemical species (e.g. methane) that are relatively catalytically non-reactive to generate electricity and heat. This simplified system optimizes fuel, electricity, and heat production, resulting in high net energy efficiencies and system flexibility.

### BRIEF DESCRIPTION OF THE DRAWING

FIG. 1 shows the process for producing liquid fuels from biomass gasification with an electrolyzer. Unit 1.1 is the biomass gasifier. Unit 1.2 is the electrolyzer. Unit 1.3 is the liquid fuel production unit. Unit 1.4 is the heating source. Stream 1.A is the biomass feed stream. Stream 1.B is the electrolyzer feed stream. Stream 1.C is the biomass gasifier product stream. Stream 1.D is the H₂ product stream from the electrolyzer. Stream 1.E is the liquid fuel production reactor feed stream. Stream 1.F is the liquid fuel production reactor product stream. Stream 1.G is the syngas product. Stream 1.H is the oxygen product stream from the electrolyzer.

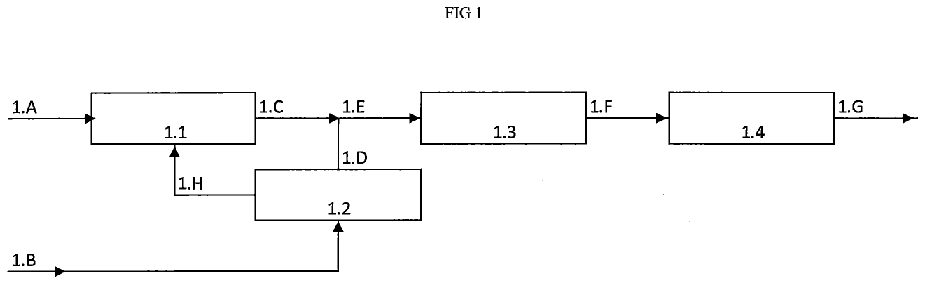

### DETAILED DESCRIPTION OF THE INVENTION

It has long been known that carbonaceous materials such as agricultural, forest and municipal waste (“biomass”) as well as natural gas, coal, oil, oil shale and oil sands (“fossil resources”) can be converted into combustible gases by thermochemical processing. These thermochemical processes utilize conversion technologies such as gasification, reforming, pyrolysis, catalysis, and other relevant processes for the conversion of fossil fuels (natural gas, coal, oil, oil shale, etc.) and renewable biomass to synthesis gas (syngas). For example, the energy options available to the world could be significantly expanded with the conversion of biomass and fossil resources to synthesis gas with further conversion to liquid fuels. Rather than burning biomass or fossil resources directly, gasification, a thermo-chemical process, is used to produce a mixture of carbon monoxide, hydrogen, and methane, known as syngas. The resulting syngas is more versatile toward end uses than the original solid biomass or fossil resources and has energy conversion possibilities that have advantages over other outputs created by direct burning.

Since the early 1900's, a number of thermochemical processes have been developed for the conversion of carbonaceous materials derived from renewable and fossil resources. These thermochemical processes convert carbon-containing compounds into syngas, a mixture comprised primarily of CO and H₂, which can also contain amounts of CO₂ and CH₄ with traces of other hydrocarbons. The technology for the production of syngas from biomass and fossil resources continues to be developed and advancements in production efficiency have been seen. Improved conversion technologies can provide benefits ranging from reduced environmental impacts to higher energy efficiency as well as secure and reliable electrical power production and fuel availability.

Biomass gasification is a chemical process that converts biological carbonaceous materials like biomass, wood, woodchips, pellets, and organic municipal solid waste into useful convenient gaseous fuels or chemical feedstock. These products are often a mixture of CO and H₂. The H₂/CO molar ratio in the product is typically less than 2.0. Pyrolysis, partial oxidation, and hydrogenation are related processes. Combustion also converts carbonaceous materials into product gases, but there are some important differences. For example, the product gas from combustion does not have useful heating value, but the product gas from gasification does. Gasification packs energy into chemical bonds while combustion releases it. Gasification takes place in reducing (oxygen-deficient) environments requiring heat; combustion takes place in an oxidizing environment giving off heat. The purpose of gasification or pyrolysis is not just energy conversion; production of chemical feedstock is also an important application. Also discovered in the early 1900's was the process for hydrogenation of CO over transition metal catalysts (the Fischer-Tropsch reaction) to produce liquid hydrocarbons and oxygenated compounds. Numerous catalyst formulations have been developed to produce liquid fuels and chemicals from syngas feedstock. For example, iron (Fe), cobalt (Co), and nickel (Ni) based catalysts have been used extensively for the synthesis of mixtures of paraffins and olefins that can be converted to gasoline and diesel fuels. Copper (Cu) based catalysts have been used for methanol and mixed alcohol production. Other metals have been proposed for deriving other specific products, for example Rhodium (Rh)-based catalysts developed for acetic acid, acetaldehyde, and ethanol production. To produce liquid fuels like diesel or sustainable aviation fuels (SAF), with a Cobalt or Nickel or Ruthenium (Ru) based catalyst, the feed to the CO hydrogenation or Fischer-Tropsch reactor typically has an H₂/CO molar ratio of 2.0 or higher. Since the primary product of biomass gasification is less than 2.0H₂/CO molar ratio in the syngas, the gasification product needs to be adjusted to greater than 2.0 molar ratio. This can be accomplished via the water gas shift reaction which converts some CO to CO₂ (see below).

Also, mixtures of catalyst metals (e.g., Rhodium and Copper) have been shown to give higher selectivity for the production of ethanol. These catalysts typically operate at elevated pressures (500-2000 psig) and temperatures (400-650° F.). The conversion efficiencies of the primary reactants (CO and H₂) to fuels can be low for alcohol synthesis (15-30%) and higher for the synthesis of chain hydrocarbons (40-80%). The amount of gas conversion decreases with gas hourly space velocity but yield and selectivity for certain products can be improved with higher space velocity.

For these synthesis catalysts, optimum space velocities are on the order of 1000 to 10,000 h-1 using normalized gas volumes.

The chemistry involved in synthesis can include the following generalized reactions.

| | | |
|---|---|---|
| CO + 3H₂ | CH₄ + H₂O | (Methanation) |
| nCO + (2n + 1)H₂ | CnH₂n + 2 + nH₂O | (Alkanes/Paraffins) |
| nCO + 2nH₂ | CnH₂n + nH₂O | (Alkenes/Olefins) |
| nCO + 2nH₂ | CnH₂n + 1OH + (n − 1)H₂O | (Alkanols/Alcohols) |
| CO + H₂O | H₂ + CO₂ | (Water-Gas Shift) |

In the synthesis of hydrocarbon and alcohol compounds with two carbons or greater, 100% selectivity for the desired compounds is not currently achievable. In addition to a mixture of liquid products, some gaseous side products are generated including carbon dioxide from the water-gas shift reaction and methane from the above methanation reaction. As described in previous systems, methane can be recycled by being reformed back to CO and H₂. Steam reforming has been described for the production of additional CO and H₂. The CO and H₂ are then added to the syngas at a point before the catalytic reactor. Since these reformers require elevated pressures and temperatures, additional external energy is needed, resulting in lower thermal energy efficiency for the production processes.

Accordingly, there is a need to identify and develop advanced technologies that will improve efficiency or reduce the cost of producing products. There is also a need to identify and develop advanced technologies that will improve efficiency or reduce the cost of producing electricity, liquid fuel, fuel gases, chemicals, and heat recovery utilities like steam within an integrated system. There is furthermore a need for a system with the capability of producing electricity, fuel gases, steam, liquid fuels and chemicals, or various combinations of these materials, while greatly reducing air pollutants and greenhouse gas emissions.

Synthesis gas is defined as a gas comprising primarily carbon monoxide (CO) and Hydrogen (H₂) from any source. Typical synthesis gas includes carbon monoxide, hydrogen, and lesser amounts of carbon dioxide (CO₂) and other useful gases such as methane (CH₄) as well as small amounts of light paraffins, such as ethane and propane. It may also contain gases such as nitrogen, argon, oxygen-containing compounds, and water in a gaseous state. However, the preferred ratio of hydrogen to carbon monoxide is 0.5 to 5, and 0.75 to 2.5 by molar ratio is particularly preferred. Accordingly, the proportion of carbon monoxide and hydrogen together in the reactant gas is preferably about 20% to about 100% by volume.

The amount of CO₂ in the syngas can also impact the synthesis activity, depending on the type of catalyst utilized. Accordingly, a preferred maximum CO₂ concentration in the syngas ranging from 0-25% is preferred, 0-10% is optimally preferred. Therefore, a system to remove some of the CO₂ from the syngas stream may be included in the syngas generation system in one embodiment. In another embodiment, waste heat is used to heat charcoal or other carbon source and carbon dioxide gas is passed over the heated charcoal to produce carbon monoxide, thereby increasing the concentration of carbon monoxide and decreasing the concentration of carbon dioxide within the syngas stream.

Catalyst contaminants like sulfur- and chlorine-containing gases, tars, and particulates may also require removal. Gas cleaning technologies like scrubbers, traps, precipitators, etc. may be included in the syngas generation system in certain embodiments.

Since syngas generation is generally an endothermic process that requires heat to produce the reaction temperatures, heat from the fuel feedstock, the generated syngas or from another external source is required to maintain a continuous reaction. The source of the heat required for the syngas generator can come from any source. In one embodiment, the reactor heat is supplied from the product gas that remains after the liquid fuel production reactions to maximize the efficiency of the fuel synthesis. In other embodiments, this heat can be supplied from the feedstock (auto thermal), generated syngas (before synthesis), or an external fuel source. The thermal efficiency of the syngas generation system should be high to maximize system efficiency and reduce parasitic load on the overall system. Thermal efficiencies of greater than 50% are preferred and greater than 70% is optimally preferred.

In one embodiment, the excess thermal energy is converted to electrical energy for use in other parts of the system or for sale on the power grid. For example, steam can be generated through a heat exchanger in the syngas outflow by either convection or conduction. The resulting steam can be used to drive a turbine or other electrical generation device as well as used in other parts of the system or sold for other industrial uses of steam.

In order to maintain the proper balance of CO to H₂ in the syngas for the desired synthesis reactions, the introduction of hydrogen to the syngas may be beneficial. In the preferred embodiment of the invention, hydrogen can be generated from electrolysis of H₂O and fed into the biomass gasifier product stream. High temperature electrolysis for the production of hydrogen gas and oxygen gas takes advantage of the waste heat of the syngas generator or other heat sources in the system. In another embodiment, hydrogen rich gas can also be generated from natural gas by steam reforming or from other emerging methods of production. Carbon monoxide concentrations of the feed gas may optionally be supplemented through carbon dioxide conversion or from an outside source.

In one embodiment of the invention, syngas and hydrogen generators are provided. In another embodiment, the syngas and hydrogen are provided from a source outside of the system. Carbon monoxide may also be supplemented in one embodiment. The system is ideally suited for small and medium sized, distributed conversion facilities that are located near feedstock resources for the production of syngas but may also be used in larger designs that may function as regional fuel and electricity production centers. Syngas, comprised primarily of CO, H₂, CO₂ and CH₄, is derived from any carbonaceous feedstock material such as agricultural, forest and municipal waste as well as natural gas, coal, oil shale and oil sands by utilizing conversion technologies such as gasification, reforming, pyrolysis, catalysis, and other relevant processes.

The syngas is pressurized and fed to a synthesis reactor to be converted to desired liquid products. Catalyst formulations are selected to convert CO and H₂ to products such as alcohols and liquid hydrocarbons. In one case, the catalyst is a supported catalyst, including cobalt, iron, or nickel deposited at between about 5 weight % and 30 weight % on gamma alumina, more typically about 20 weight % on gamma alumina, based on the total weight of the supported catalyst. Also, the supported catalyst formulation includes selected combinations of one or more promoters consisting of ruthenium, palladium, platinum, gold, nickel, rhenium, and combinations thereof in about 0.01-20.0 weight % range, more typically in about 0.1-0.5 weight % range per promoter. The liquid products are cooled and separated from the un-reacted gases. Catalyst formulations are also selected to convert CO and H₂ in the syngas to the desired fuel products such as alcohol and liquid hydrocarbon fuels without significantly altering the more energy rich chemical species such as methane and other catalytically, non-reactive species in the syngas.

The desired liquid products are separated from the energy enriched gas stream and an on-line process analyzer is used to monitor the concentrations of the primary gas species. This information, along with process control algorithms, is used to adjust the amount of gas that is recycled through the catalyst versus the amount of gas that is sent to the engine/generator or gas-turbine. This integrated, continuous process results in an optimized production of liquid fuels, electricity, and heat.

A portion of the product gas and the electricity generated by the system may be used to provide process energy for various parts of the system including the syngas generator, a hydrogen generator and other equipment described in detail below. An analog and/or digital control system optimizes the gas composition for production of fuels and electricity.

Some of the electricity and heat may be used to produce hydrogen, which may be added to the incoming syngas to increase the yield of liquid fuel products. The composition of the syngas stream may be further supplemented by passing carbon dioxide gas over heated charcoal to form carbon monoxide increasing the concentration of this gas to the reactor. Hydrogen gas may also be added to bring the concentrations of CO and H₂ to the desired ratio.

The electricity and heat generated in this system may also be used to run the process and it may also be used as a clean energy source for the syngas production systems. Excess electricity may be distributed to the regional electrical grid. The excess heat can be used for other co-located processes and facilities.

In one embodiment, a system for the production of liquid fuels and electricity from syngas is provided that 1) generates synthesis gas (syngas) from a carbonaceous feedstock (e.g. agricultural, forest and municipal waste, natural gas, coal, oil shale and oil sands) using a thermochemical process, catalytic reformer and/or other relevant technology or combination thereof; 2) introduces a clean syngas stream (primarily CO, H₂, CH₄, CO₂ and H₂O at varying concentrations) to a catalytic synthesis reaction system to simultaneously generate liquid fuel from CO and H₂ while concentrating CH₄ and other combustible but non-reactive gases in a gas product stream; and 3) introduces the CH₄ rich product gas stream into an engine, gas-turbine or other relevant technologies for the production of electricity and heat.

In one embodiment, a system is provided where catalyst formulations in the synthesis reactor are selected to operate under conditions in which CO reacts efficiently with H₂ to produce alcohol, diesel, gasoline, ether, or hydrogen fuels as well as create conditions in which CH₄ and other light hydrocarbons are relatively non-reactive over the catalysts.

Process optimization is optionally facilitated by the injection of additional hydrogen into the catalyst reactor produced by electrolysis or other source to improve liquid fuel production efficiency. The process control computer is used to change the proportion of liquid fuel production to electricity/heat production and dictated by real-time electricity and liquid-fuel market fluctuations and requirements. In one embodiment, extra heat from the electrolyzer is used to preheat the liquid fuel production reactor product to a temperature of greater than 150 F.

In one embodiment, a system is provided that monitors the composition of the pre- and post-catalyst gas stream and uses that information with an on-line process controller to optimize the recycle ratio of the post-catalyst gas stream and other process controls with the purpose of generating a CO and H₂ rich gas stream suitable for fuel synthesis via catalysis, as well as a CH₄ and other light combustible rich gas stream that is ideal for the production of electricity and heat.

In one embodiment, a controller wherein chemical thermodynamic and energy models and/or advanced learning algorithms (e.g., neural networks) are used in an on-line digital and/or analog process control system to help determine the desired process conditions and split ratios of the gas stream to the catalysts and the engine/generators and/or gas turbine/generators.

In one embodiment, the energy efficiency of the process is maximized by monitoring the pre- and post-catalyst gas stream composition and using that information in an on-line process control system to vary fuel production vs. electricity and heat production. The on-line process control system preferably employs kinetic/thermodynamic models and neural network systems.

In one embodiment, sensors monitor the gas composition at one or more locations, in combination with chemical thermodynamic and energy models to control the addition of the hydrogen into the pre-catalyst syngas stream to enhance the catalyst conversion efficiencies of CO, CO₂ and other catalytically reactive species to fuels.

In one embodiment, a process control system is used to change the proportion of fuel production to electricity and heat production, depending upon real-time market conditions. For example, when there is a peak load demand for electricity, the amount of electricity can be increased to help meet that demand.

In one embodiment, the process of biomass gasification and green hydrogen from water electrolysis are integrated such that there is no need for the Water Gas Shift reaction. The electrolysis of water produces hydrogen that is added to the biomass syngas such that the total volume of H2+CO is greater, and the overall plant production rate is increased by 20-1000% of fuels, or 30-90% of the volume of the electrolyzer only case or even 40-80% of the no biomass gasification case.

In one embodiment, the oxygen produced by the electrolyzer is used in the biomass gasifier. This replaces the need for an Air Separation Unit leading to improved economics and higher overall energy efficiency for the case with biomass gasification alone. The electrolysis of water produces oxygen that is added to the biomass gasifier such that the total volume of H2+CO is greater, and the overall plant production rate is increased by 20-1000% of fuels, or 30-90% of the volume of the electrolyzer only case or even 40-80% of the no biomass gasification case.

In one example, the biomass gasifier's output was 100 kmol/hr at an H₂/CO molar ratio of 0.742. The H₂ output was 28.75 kmol/hr and the CO output was 38.75 kmol/hr. H₂ from the electrolyzer was added to the biomass gasification product stream to reach a target H₂/CO molar ratio of 2.1. This led to an increase in syngas to 120.125 kmol/hr. Without the added H₂, the result would be 67.5 kmol/hr of syngas. Using this method led to an increase in production by 78%. This leads to an increase in the total fuel production by combining the electrolyzer hydrogen with the H₂ and CO produced by the biomass gasification.

In one embodiment, a non-thermochemical process is used to produce additional syngas. Non-thermochemical processes to produce syngas use biological organisms, including bacteria and yeast to produce syngas. In one embodiment, the biological process is ABE conversion to syngas. Acetone-butanol-ethanol (ABE) fermentation, also known as the Weizmann process, is a process that uses bacterial fermentation to produce acetone, n-butanol, and ethanol from carbohydrates such as starch and glucose that are converted to syngas.

In one embodiment, a non-thermochemical and biological process to produce ethanol that can be converted to hydrocarbons, jet fuel, or syngas. Ethanol fermentation is a biological process which converts sugars such as glucose, fructose, and sucrose into cellular energy, producing ethanol and carbon dioxide as by-products. Because yeasts perform this conversion in the absence of oxygen, alcoholic fermentation is considered an anaerobic process. In one embodiment, the ethanol is further converted to hydrocarbons via a dehydration catalyst that removes the oxygen to produce ethylene. In one embodiment, ethylene is catalytically oligomerized to long chain hydrocarbons. The long chain hydrocarbons are used as renewable diesel and sustainable aviation fuel in the overall process.

1. A process for the production of liquid fuels, wherein the process comprises:
a) generating synthesis gas from a carbonaceous feedstock using a biomass gasifier to provide a first product stream;
b) feeding water into an electrolyzer to provide a second product stream comprising hydrogen and a third product stream comprising oxygen;
c) combining the first product stream and the second product stream to provide a first feed stream;
d) feeding the first feed stream to a liquid fuel production reactor comprising a catalyst that catalyzes the conversion of syngas to liquid fuel
thereby producing liquid fuels.

2. The process of claim 1, wherein the synthesis gas comprises CO, H₂ and combustible gases, and wherein the combustible gases are concentrated into a combustible gas stream, and wherein the combustible gas stream is used for the production of heat or electricity.

3. The process of claim 1, wherein the third product stream is fed to the biomass gasifier as an oxidant.

4. The process of claim 1, wherein there is an amount of liquid fuels produced, and wherein combination of the second product stream with the first product stream increases the amount of liquid fuels produced, as compared to a process where only the first product stream is fed into the liquid fuel production reactor, by an amount ranging from 10 percent by volume to 100 percent by volume.

5. The process of claim 2, wherein the combustible gas stream is used for the production of heat, and wherein the heat is used for the conversion of syngas to liquid fuel.

6. The process of claim 2, wherein the combustible gas stream is used for the production of electricity, and wherein the electricity is used for running the electrolyzer.

7. The process of claim 3, wherein there is an amount of liquid fuels produced, and wherein feeding the third product stream to the biomass gasifier increases the amount of liquid fuels produced, as compared to a process where the third product stream is not feed to the biomass gasifier, by an amount ranging from 10 percent by volume to 100 percent by volume.

8. A process for the production of liquid fuels, wherein the process comprises:
a) generating synthesis gas from a carbonaceous feedstock using a biomass gasifier to provide a first product stream;
b) generating synthesis gas using a non-thermochemical process to provide a second product stream;
c) combining the first product stream and the second product stream to provide a first feed stream;
d) feeding the first feed stream to a liquid fuel production reactor comprising a catalyst that catalyzes the conversion of syngas to liquid fuel
thereby producing liquid fuels.

9. The process of claim 8, wherein there is an amount of liquid fuels produced, and wherein combination of the second product stream with the first product stream increases the amount of liquid fuels produced, as compared to a process where only the first product stream is fed into the liquid fuel production reactor, by an amount ranging from 10 percent by volume to 100 percent by volume.

10. The process of claim 8, wherein the process further comprises feeding water into an electrolyzer to provide a third product stream comprising hydrogen and a fourth product stream comprising oxygen, and wherein the first feed stream is combined with the third product stream to provide a second feed stream, and wherein the second feed stream is fed into the liquid fuel production reactor.

11. The process of claim 10, wherein the synthesis gas comprises CO, H₂ and combustible gases, and wherein the combustible gases are concentrated into a combustible gas stream, and wherein the combustible gas stream is used for the production of heat or electricity.

12. The process of claim 11, wherein the combustible gas stream is used for the production of heat, and wherein the heat is used for the conversion of syngas to liquid fuel.

13. The process of claim 11, wherein the combustible gas stream is used for the production of electricity, and wherein the electricity is used for running the electrolyzer.

14. A system for the production of liquid fuels, wherein the system comprises: a biomass gasifier operably connected to an electrolyzer, such that a first product stream from the biomass gasifier can be combined with a second product stream from the electrolyzer in a chamber to provide a first feed stream; a liquid fuel production reactor operably connected to the chamber, such that it can receive the first feed stream.

15. The system of claim 14, wherein the electrolyzer is a solid oxide electrolyzer.

16. The system of claim 14, wherein the system further comprises a conduit for the delivery of oxygen from the electrolyzer to the biomass gasifier.

17. The system of claim 14, wherein the system further comprises a concentrator operably connected to the biomass gasifier for the concentration of combustible gases produced by the biomass gasifier.

18. The system of claim 17, wherein the system further comprises a conduit operably connected to the concentrator, and wherein the conduit is further operably connected to a gas turbine for the production of electricity.
